(19) United States
(12) Patent Application Publication
(10) Pub. No.: US 2002/0022799 A1
(43) Pub. Date: Feb. 21, 2002

(54) Enhanced energy balloon catheter

(76) Inventor: Marc G. Apple, Fort Wayne, IN (US)

Correspondence Address: Anton P. Ness, Patent Attorney, P.O. Box 2269, Bloomington, IN, 47402-2269, US

(21) Appl. No.: 09/748,327

(22) Filed: Dec. 26, 2000

Related U.S. Application Data
(60) Provisional application No. 60/173,266, filed on Dec. 28, 1999.

(51) Int. Cl. A61M 29/00

(52) U.S. Cl. 604/96.01

(57) ABSTRACT

A balloon catheter assembly (10) including a balloon section (14), a radiofluid port (40), and a magnetizing section. The magnetizing section generates a magnetic field that enhances the energy of the electrons (Beta particles) from radiofluid within the balloon section to increase the depth dose. The magnetizing section may comprise a solenoid arrangement (70) wherein wires (72,74) within catheter wall (20) having coils (76) surrounding the catheter within the balloon section (14) are connected to a controlled source (86,88) of milliamperage or microamperage, and generate a magnetic field within balloon section (14). Permanently magnetized distal end portions (116,138) of the catheter (112) or guide wire (118) or both, within balloon section (114), may be used to generate the magnetic field, or may be used in conjunction with a solenoid arrangement.

### CROSS-REFERENCE TO RELATED APPLICATIONS

This application claims priority of provisional application Serial No. 60/173,266 filed Dec. 28, 1999.

### TECHNICAL FIELD

This relates to the field of medical devices and more particularly to balloon catheters.

### BACKGROUND OF THE INVENTION

It is known to utilize balloon catheters for angioplasty, or reducing the effect of atherosclerotic plaque on and intraluminal narrowing of the arterial walls within the vascular system of a patient. The balloon portion of the catheter is then inflated to a predetermined pressure range and size, to radially compress the plaque occlusion, thereby increasing the internal diameter of the previously restricted artery, after which the balloon is collapsed and the catheter removed. However, after the angioplasty procedure has been performed, as many as one-third to one-half of the patients soon develop restenosis. Restenosis can occur after angioplasty or other recannulation procedures, with or without stenting, wherein the migration and proliferation of benign cells cause a restenotic lesion to form, resulting in the further blockage of the intravascular structure.

It is known from U.S. Pat. Nos. 5,059,166; 5,213,561; and 5,302,168 to administer radiation to patients for a variety of reasons, such as to treat restenosis, malignant or benign tumors, or the like.

It is known from U.S. Pat. No. 5,916,143 and U.S. patent application Ser. No. 09/253,310 filed Feb. 20, 1999 to utilize a balloon catheter for delivery of a contained therapeutic dosage of radiofluid to the lesion site, for prevention or minimization of restenosis or for treatment of a tumor, for example. After treatment, the radiofluid is recovered from the patient. One preferred radiofluid is xenon-133 inert gas. The radiation dosage is precisely measured and controlled, while shielding material of the catheter wall at all locations except the balloon at the lesion site, protects healthy tissue from the radiation; additional shielding or attenuation is provided by the catheter system to protect the medical personnel.

In radiotherapy, a dose of radiation is prescribed by the treating physician and represents the total cumulative ionizing radiation energy deposition that is expected to be minimally delivered to the tissue layer depth or a target lesion in order to achieve an expected clinical outcome as a result of known radiobiological effects for that radiation dose to that specific tissue. The dose delivered from xenon-133 by such a balloon catheter system emits at least 90% of its energy within the first 0.6 mm of tissue measured from the balloon's outer surface, due to beta particle production by the xenon-133 gas. Some contribution exists from the emitted X-rays that are more important in deeper tissue depths.

Therefore, the amount of energy deposited or delivered is a direct product of the amount of initial radioactivity in the system (millicuries), the peak and average energies of the emitted beta particles, and, partially, the percent and net energies of emitted X-rays. This will provide a xenon-133-specific and balloon size-specific depth dose rate.

It is desired to provide enhanced benefit of treatment by radiofluids while not increasing the amount, or while even decreasing the amount, of net radionuclide activity, or the amount of radiofluid being administered, or the length of time of administration.

### SUMMARY OF THE INVENTION

The foregoing problems are solved and a technical advance is achieved in the energy enhancement system of the present invention. In a first aspect, a catheter comprises a system for delivering treatment material to a treatment site in a patient, the treatment material providing energetic electrons to the treatment site. The catheter also provides a magnetic field at the treatment site to enhance the energetic electrons to relatively higher energy levels for more effective treatment. A balloon arrangement can be utilized at the treatment site to move the electron source treatment material close to or against the vessel wall.

In one particular embodiment that utilizes radioactive fluid to inflate a balloon at the treatment site, where the radioactive fluid is a source of the energetic electrons, the present invention provides a magnetic field upon a selected region of the catheter wall, such as the balloon wall and/or the space within an inflated balloon, in a balloon catheter adapted for administration of radiofluid to a treatment site within a patient. Either a static or a low-voltage electroinduced magnetic field is useful. The radiofluid may be, for example, xenon-133 or xenon-127. Decaying xenon-133 gas produces a constant spectrum of energetic electrons up to 364 keV with a known baseline average and peak depth of radiation dose distribution for tissue density; the present invention may however be used with other radiofluids or inert nonradioactive fluids. By providing the magnetic field to the energetic electrons emitted, creation of single or mixed polar field of magnetism immediately adjacent to the already energetic electrons for a short distance within the balloon (such as 0.5 to 1.0 mm) can directionally shift and concentrate the electrons outwardly toward the desired tissue surface and thereby predictably change the depth dose and increase the dose rate. The strength of a predetermined magnetic field is selectively controlled immediately around the catheter wall within the balloon, thereby precisely controlling the extent of the shift in relative depth dose, to produce an increased yield of deposited dose desired to tissue or a target volume/area, for a given dose. Also, there can be an additional yield of short distance penetrating photons into the target tissue as a byproduct of bremsstrahlung energy as the plentiful lower energy electrons interact with the metal density of the magnet(s).

The fixed, pre-formed and constant magnetic field can easily be integrated into a selected region of a catheter wall by several structural mechanisms or binding processes. Materials are available commercially in varying strengths, shapes, and sizes, and can be custom-molded. One catheter with which the present invention may be used, is under clinical and commercial development as the Xenacath® catheter produced by Cook Incorporated, Bloomington, Ind.

In one embodiment, a pre-made port sealed gas-tight catheter could have an attachable plug adjacent to or within the port whereby a two-wire connection point can be connected to a small transportable milliampere or microampere source, such as a battery. An ultrathin insulated wire extends from the connection point to the balloon and defines several coils around the intra-balloon catheter wall. With the port component being connected to both the radiogas injector and the battery connector, a pre-set milliampere or microampere supply is then turned on which produces flux-varying micromagnetic fields for a short distance within the balloon. When the radiogas is injected, the constant supply of electrons produced via radiodecay are immediately accelerated to desired additional average and peak keV energy levels affecting only the electrons within the balloon while increasing the energy yield and penetration depth of the beta particles. Direct or alternating low amperage currents through such a wire and at the segment with the multiple wire coil turns, potentially around a small magnet-containing segment of the intra-balloon catheter, will create polar magnetic fields that will accelerate and/or repel already energetic/moving electrons to higher energies depending on their starting energies. When treatment is complete, the battery supply is turned off, the energy emission of limited activity radiogas in the balloon returns to original energy levels, and the gas is withdrawn and catheter disposed of in routine fashion. Potentially much less radiogas activities, diminished treatment times and idealized yet radiosafe depth dose rates could be achieved as needed.

In a second embodiment, a segment of the guidewire adjacent to or within the distal end of the catheter is permanently magnetized, thus generating a magnetic field at the treatment site.

In another embodiment, a fixed magnetic component is affixed onto or embedded within the catheter's outer surface adjacent the distal end, providing a magnetic field at the treatment site of fixed polarity.

Optionally, several magnetic fields may be established axially spaced within the balloon, by variable polarized “magnetized” cuffs of permanent magnetic material around the catheter wall or within the wall, contained within the balloon.

Additionally, an embodiment can utilize a catheter assembly as in the first embodiment, having catheter-contained coils of a wire connected to a battery source, may be used in conjunction with either a guide wire with magnetized distal end portion, or with a magnet-containing catheter distal end portion.

Embodiments of the present invention will now be described by way of example with reference to the accompanying drawings.

### BRIEF DESCRIPTION OF THE DRAWINGS

FIG. 1 is a schematic elevation view of a balloon catheter with a solenoid embodiment of the present invention;

FIG. 2 is an enlarged cross-sectional view of a section of the catheter of FIG. 1 after inflation of the balloon at a treatment site within a vessel of a patient;

FIG. 3 is an elevation view similar to FIG. 1 showing an embodiment of a catheter and an associated guide wire each with a permanently magnetized distal end portion;

FIG. 4 is an enlarged cross-sectional view of a section of the catheter of FIG. 3 after balloon inflation showing the magnetic flux and resultant effect on the paths of representative electrons of the radiogas; and

FIG. 5 is an enlarged longitudinal section view of the inflated balloon section of an alternate embodiment illustrating several magnetic fields spaced axially along the balloon.

### DETAILED DESCRIPTION

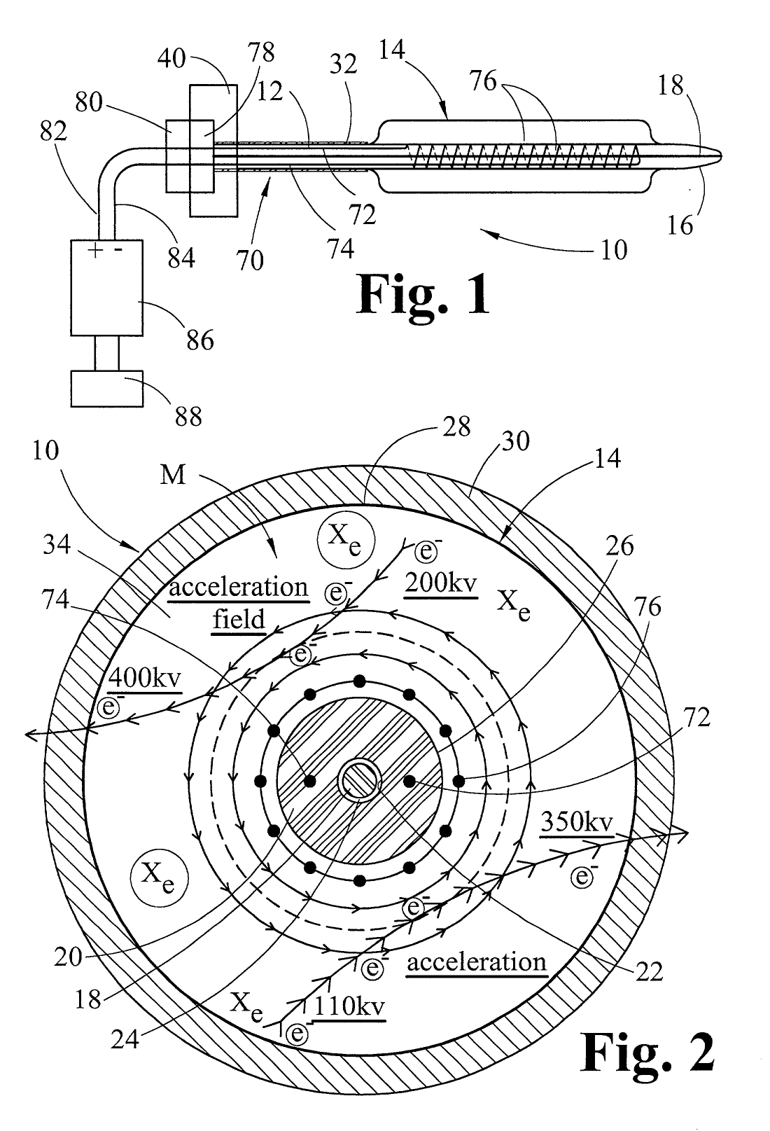

FIGS. 1 and 2 illustrate a preferred embodiment of the present invention. Catheter assembly 10 includes a catheter 12, a balloon section 14 at a distal end 16, a guidewire 18, and a radiofluid port 40 whereinto the radiofluid is to be injected, with an inflation lumen extending from the port to the balloon at the balloon section. With reference to FIG. 2, catheter wall portion 20 within balloon section 14 includes an inner wall surface 22 defining a central lumen 24, and an outer wall surface 26. Guidewire 18 is disposed within central lumen 24, and balloon section 14 surrounds outer catheter wall surface 26. Balloon section 14 is shown in its inflated state, with balloon wall 28 adjacent to and pressing against vessel wall 30 of a patient as during treatment. Such a catheter with a radiofluid port 40 is described in greater detail in U.S. Pat. No. 5,916,143 and U.S. patent application Ser. No. 09/253,310. Catheter may be fabricated from any of several well-known biocompatible materials, such as polyethylene, and balloon section 14 may also comprise polyethylene. The balloon wall thickness is preferably from about 70 to about 200 μm. Catheter 12 is shielded at all locations along its length except at balloon section 14, by shielding material 32, such as tungsten or lead alloy equivalents.

Magnetizing section 70 includes wires 72,74 having a plurality of coils 76 situated adjacent the distal catheter end 16 radially within the balloon section 14. Coils 76 are shown surrounding outer catheter wall surface 26. Wires 72,74 extend to connector port 78 at a proximal end of catheter 12, where they are interconnected by means of a mating connector 80 to wires 82,84 that extend to a low amp oscillator 86 and to a battery source 88. Wires 72,74 may be a gauge similar to that of a guide wire that is appropriate for the particular vascular or organ treatment site, such as about 0.0014 to 0.0018 in, for example.

Beta particles essentially are random, non-current generating, moving electrons produced at fixed kinetic energy spectra from radioactive decay of the radiogas, due to the characteristics of radiodecay for that gas. As such they are manipulatable with magnetic fields because of their negative charge. Higher energy beta particles emitted from types of radionuclides other than xenon-133, can provide a greater tissue energy deposition and to a deeper depth because of the inherent principles of physics for beta particles and the higher initial kiloelectrovolts (keV) of the particles, for the same relative activity in millicuries (the number of atoms undergoing radiodecay per second).

Being essentially electrons, the beta particles can be directionally repelled toward tissue surfaces, or accelerated in velocity, or both, by a fixed net negative same-pole magnetic field, or a fluctuant mixed polarity magnetic field. It is known that when electrons are subjected to a short distance magnetic flux within a positive polarity field, the electrons can be accelerated to a higher kinetic energy depending on the strength of the magnetic field flux, the proximity of the electron to it, and the relative time of exposure to the field prior either to collision with other particles or to emission of the beta particles into the tissue.

Therefore, if an average known quantity of magnetic flux per distance (in millimeters) is exposed to a known activity of xenon-133 beta particles, the average energy of the emitted beta particles will be increased, proportional to the magnetic field flux strength, such that they will possess a regulatable greater dose deposition and to a greater tissue depth while exposed to the magnetism. X-rays or photons are non-charged energy emissions and would not practically be affected by such magnetic fields.

As shown in FIG. 2, interior balloon region 34 is filled with radioactive fluid containing for example xenon-133 or xenon-127 (designated as “Xe”), or a mixture of both. Seen within catheter wall 20 are wires 72,74 that extend to the balloon section and then extend to outer wall surface 26 to define coils 76. Power from power source 88 is accessed to generate an electrical current in coils 76, thereby generating a magnetic field M. When balloon 14 is inflated with radioactive fluid, the beta particles or electrons from the nuclear decay of the radioactive gas are influenced by the magnetic field. As shown in FIG. 2, both the direction and the energy level of an electron is enhanced by the magnetic field. Each free electron is repelled by the magnetic field to pass through balloon wall 28 and enter patient tissue at the treatment site.

Representative free electrons are shown (designated as “e⁻”), with their initial energy levels, their paths as they enter and are deflected by the magnetic field, and their exit levels. One representative electron is shown with an initial level of 110 keV and an exit level of 350 keV, while another is shown with an initial level of 200 keV and an exit level of 400 keV, all due to a net acceleration from the magnetic field. The average higher energies and depth doses of the electrons are predictable from the strength of the flux of a low amperage electro-magnetic field. Photons, as X-rays and gamma rays also produced by the radioactive fluid, are not directly influenced by the magnetic field.

At present, the average electron energy for xenon-133 radiation treatment, is about 110 keV, with a peak energy of about 360 keV. As a result of energy enhancement with the present invention, the average electron energy is shifted from 110 keV to 200 or 300 keV resulting from enhancement. Electrons whose energy is enhanced to 400 keV are expected to penetrate 1mm deep into tissue, as opposed to only 0.8 or 0.9mm depth for 360 keV electrons; also, more electrons will be at the higher energy levels, so more electrons will quantitatively penetrate patient tissue.

The field magnetic flux and intensity will be proportional to the number of wire turns and the radius, as well as the nominal amperage. The number of loops in the wire coil may range from about 3 to about 200 or more depending on the type of wire, metal, density, thickness and impedance. It will also be affected by the presence of any fixed or static magnets, fluctuation of amperage through the wire, and relative radius of intended effect, all of which would be relatively very small and innocuous to deeper adjacent tissues.

The strength of the magnetic field may be calculated according to primary applications or variations of one of the following formulas:

$$B = \frac{{μ}_{o} iN}{2 π ( r )}$$

(for infinite straight filament or solenoid)

$$B = \frac{{μ}_{o} {ir}^{2}}{2 {( {r}^{2} + {x}^{2} )}^{3 / 2}}$$

(for circular current loop)

where:

B=magnetic field (in Teslas)

μₒ=permeability constant (1.26×10⁻⁶ H/m)

i=current in wire (in amperes)

N=number of turns per unit length of solenoid (in 1 /meter)

r=radius of current loop (in meters)

x=distance, on axis, from center of current loop (in meters)

The range of electrical current may be selected to result in the preferred minimal flux density for desired radius effect of at least 4000 to 12000 gauss with magnetic field strengths of at least 3 to 35 Megagauss.

The strength of the magnetic flux, and thereby the acceleration energy of the beta emissions, is a controllable consequence of the type of wire or conductive material, the amperage of applied field current, the number of rotations of wire creating the field, and the average radial distance of the affected electrons from the surface of the magnetic field, and the application time of alternating or direct current. This can be quantitatively interpreted according to the previously described formulas. For the same activity of xenon-133 radiogas, one may yield greater overall total dose delivery and to a deeper tissue depth for a given unit of exposure time due to the conversion of magnetic field energy to ionizing radiation energy by increasing the net average kinetic energy of a radiodecay-created beta particle.

In the embodiment shown in FIG. 3, the catheter wall portion 120 of catheter 112 adjacent to distal end 116 within the balloon section 114, contains a permanently magnetized outer surface 126 such as of plating material such as, for example, ferrite, AINiCo or rare earth metals; optionally, the catheter wall can be impregnated with particles of magnetized metal 142. Guide wire 118 is shown to include a magnetized distal end portion 138 with a magnetized length of 2 to 8 cm for vascular applications and 4 to 10 cm for other applications. The magnetized material may be for example, ferrite, ALNiCo or rare earth metals. One or both permanently magnetized catheter assembly portions may be utilized to generate a magnetic field within the balloon portion 114. One or both may also be utilized in conjunction with the solenoid embodiment of FIGS. 1 and 2.

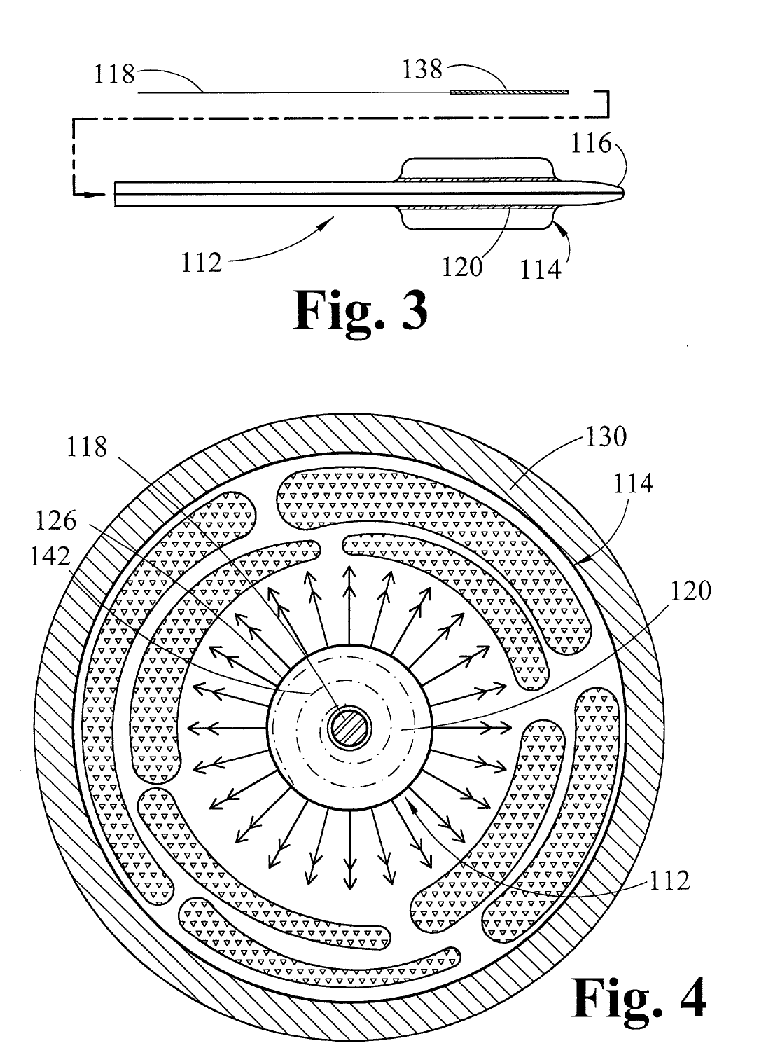

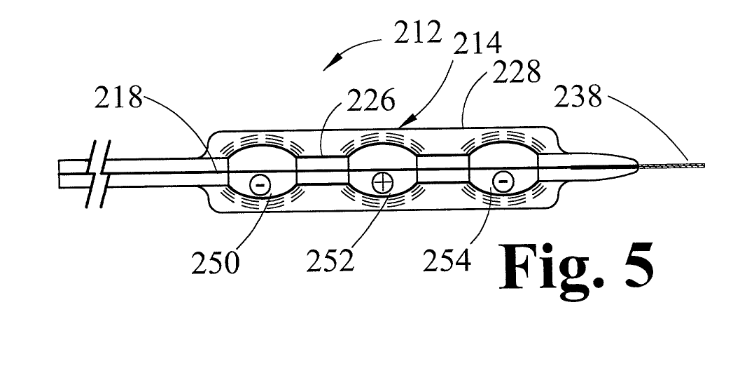

FIG. 4 illustrates in cross-section a catheter 112 with an expanded balloon section 114 within a vessel wall 130, where catheter wall portion 120 of catheter 112 is permanently magnetized. A unipolar (negative) magnetic field is generated by the magnetic material, that has a short distance radially outwardly from the catheter outer wall.

Optionally, as shown in FIG. 5, the catheter may have a plurality of permanently magnetized sections, all within the balloon section. In catheter 212, balloon section 214 encloses three permanently magnetized sections or cuffs 250,252,254 of the catheter, spaced axially therealong; more such sections may be used if desired. Alternating ones are polarized oppositely: sections 250 and 254 for example may be negative while intermediate section 252 is polarized positively. The arrangement is analogous in concept to a linear accelerator, and would have the effect of accelerating the particles. Each cuff may be defined by either magnetic plating material on the outer surface 226 of the catheter wall, or by particles embedded within the wall. Optionally, also, guide wire 218 may have a permanently magnetized distal end portion 238, similarly to FIG. 3. Balloon wall 228 may have its thickness or density increased to be permeable only to higher energy particle emissions, and to be impermeable to low energy particle emissions that otherwise would have only resulted in excessive superficial dosing.

The present invention enhances the energy of the electrons emitted by radiodecay and would have the advantage of either permitting a deeper dose, or reducing the time of exposure, or using less radiogas. Anatomic sites other than intravascular sites, with an appropriate balloon diameter and length to allow for tissue abutment, include the esophagus, the upper or lower gastrointestinal tract, the biliary duct system, gynecological tissues such as the vaginal vault and cervix, the lung and tracheo-bronchial system, the oral-nasopharyngeal cavities, the bladder and genito-urinary collection system, or in post-operative sites of the brain, soft tissue, the liver and muscles, and so forth. Malignant or non-malignant dose radiotherapy may be made more efficacious or practical due to the improved depth dose profile benefits and controlled radiation dosing with brachytherapy as applied using a magnetized or magneto-enhanced inert radiogas/radiofluid delivery system.

It is thus seen that the present invention has utility in a variety of medical procedures, and variations and modifications of the enhanced energy balloon catheter of the present invention additional to the embodiments described therein are within the spirit of the invention and the scope of the claims.

What is claimed is:

1. A catheter for administration of relatively high energy electrons to a treatment site of a patient, said catheter comprising a balloon arrangement to which treatment material can be applied, said treatment material being capable of producing energetic electrons, said catheter also comprising in the region of the balloon arrangement at least one magnetizing section for enhancing said energetic electrons to said relatively high energy electrons.

2. A catheter according to claim 1, wherein the or each magnetizing section is arranged to provide a radially directed magnetic field.

3. A catheter according to claim 1, wherein the or each magnetizing section is arranged to provide an axially orientated magnetic field, and wherein means are provided for deflecting enhanced electrons toward said treatment site.

4. A catheter according to claim 3, wherein said means for deflecting comprises a magnetizing section of a polarity opposite to that of an adjacent section.

5. A catheter according to claim 1, wherein the or each magnetizing section comprises permanent magnets, or wherein the or each magnetizing section comprises an energizable coil or coils, or wherein a plurality of sections comprises a combination of energizable coils and magnets.

6. A catheter for administration of enhanced high energy electrons to a treatment site of a patient, said catheter comprising at least one balloon section located at a distal region of the catheter, said catheter being connectable to means for inflating the at least one balloon section so that when inflated there is contained treatment material capable of emitting high energy electrons, said catheter also comprising within or in the region of each balloon section at least one magnetizing arrangement for increasing the energy of the high energy electrons and for directing the enhanced high energy electrons to the treatment site.

7. A catheter according to claim 6, wherein each magnetizing arrangement comprises either one or more permanent magnets, one or more energizable coils, or a combination of one or more permanent magnets and one or more energizable coils.

8. A system for the administration of enhanced high energy electrons to a treatment site of a patient, said system comprising a catheter according to claim 1, means for connecting the catheter balloon to a source of the treatment material, and a control arrangement for controllable activation and deactivation of at least one magnetizing section or arrangement.

9. A balloon catheter for administration of radiofluid to a treatment site of a patient, comprising:
a catheter having a balloon section;
a radiofluid port and passageway extending from said port to said balloon section for balloon inflation thereat; and
at least one magnetizing section for establishing a magnetic field within the balloon section for enhancing the energy of the radiofluid.

10. The balloon catheter as set forth in claim 9 further including a control for controllable activation and deactivation of said at least one magnetizing section wherein said control is connected to said radiofluid port for precisely coordinating the activation and deactivation of said magnetizing section with flow of said radiofluid.

11. The balloon catheter as set forth in claim 9 wherein several magnetizing sections are spaced apart axially within the balloon section.

12. The balloon catheter as set forth in claim 9 wherein said magnetizing section includes a wire extending along and within said catheter to a distal wire end including at least one coil surrounding an outer wall of a portion of said catheter disposed within said balloon section, and further includes a source of electrical current connected to a proximal end of said wire and a control for activating said electrical current.

13. The balloon catheter as set forth in claim 12, further including a connector between said source and said wire.

14. The balloon catheter as set forth in claim 13 wherein said connector is removable and reconnectable to said catheter assembly at a port thereof, to disconnect and reconnect said source, respectively.

15. The balloon catheter as set forth in claim 12 wherein said wire is 12 gauge wire or smaller.

16. The balloon catheter as set forth in claim 12 wherein said source is a battery producing alternating or direct current of microamperes or milliamperes of electrical current within confined pathways.

17. The balloon catheter as set forth in claim 9, further including a guidewire.

18. The balloon catheter as set forth in claim 17 wherein said guidewire includes a distal end portion coextendable along said balloon section, and said distal guidewire end portion includes magnetized material.

19. The balloon catheter as set forth in claim 9 wherein said catheter includes a distal end portion coextendable along said balloon section, and said distal catheter end portion includes magnetic material therearound.

20. A method of therapy, comprising:
providing a balloon of a balloon catheter at a treatment site in a vessel of a patient;
injecting radiofluid into said balloon, thereby expanding said balloon;
inducing a magnetic field onto said radiofluid for a selected length of time, thereby enhancing an energy of said radiofluid and increasing a therapeutic effect thereof; and
withdrawing said radiofluid.

21. The method as set forth in claim 20 wherein said balloon catheter includes a wire coil connected to a control, and said inducing of a magnetic field comprises activating an electrical current in said wire coil.
